(19) United States
(12) Patent Application Publication
(10) Pub. No.: US 2010/0217299 A1
(43) Pub. Date: Aug. 26, 2010

(54) SURGICAL REMOVAL OF INTERNAL TISSUE

(76) Inventors: Zev Williams, Brookline, MA (US); Alexander H. Slocum, Bow, NH (US); Christopher Y. Brown, Olney, MD (US); Darragh Buckley, Ann Arbor, MI (US); Daniel Hernandez-Stewart, Naples, FL (US); Aparna Rolfe, Newton, MA (US); Samuel Kesner, Needham, MA (US)

Correspondence Address: NUTTER MCCLENNEN & FISH LLP, SEAPORT WEST, 155 SEAPORT BOULEVARD, BOSTON, MA, 02210-2604, US

(21) Appl. No.: 12/529,082

(22) PCT Filed: Mar. 5, 2008
(86) PCT No.: PCT/US08/55907

§ 371 Date: May 11, 2010

Related U.S. Application Data
(60) Provisional application No. 60/904,977, filed on Mar. 5, 2007.

(51) Int. Cl. A61B 17/32

(52) U.S. Cl. 606/180

(57) ABSTRACT

Methods and devices are provided for macerating and removing tissue. In general, a maceration device is provided that can be distally advanced into a body in a minimally invasive surgical procedure and positioned proximate to tissue desirable for removal from the body. The maceration device can include an elongate shaft having a cutting element positioned on the shaft's side (i.e., not located on a distal tip of the elongate shaft). The cutting element can rotate to macerate tissue. When being introduced to the body, an elongate axis of the elongate shaft and a longitudinal axis of the cutting element can be substantially parallel to each other. When the cutting element rotates, the elongate axis of the elongate shaft and longitudinal axis of the cutting element can not be parallel during at least a portion of the cutting element's rotation.

### CROSS-REFERENCE TO RELATED APPLICATIONS

This application claims priority to U.S. Provisional Patent Application No. 60/904,977 filed on Mar. 5, 2007 and entitled “Device For The Minimally Invasive Surgical Removal Of Internal Tissue,” which is hereby incorporated by reference in its entirety.

### FIELD OF THE INVENTION

The present invention relates to methods and devices for removing internal tissue, and in particular to methods and devices that are effective to macerate and remove tissue from a body.

### BACKGROUND OF THE INVENTION

A hysterectomy is the surgical removal of part of or the entire uterus. Hysterectomies are the most common gynecological surgeries performed in the United States, with 600,000 procedures performed every year. Laparoscopic hysterectomy is the removal of the uterus through a small incision after surgically separating the uterus from the cervix and fallopian tubes and cutting the uterus into manageably small pieces. Laparoscopic hysterectomies currently take longer to perform than abdominal hysterectomies but result in less postoperative pain, shorter length of hospitalization, quicker recovery, and better quality of life six weeks post operation.

Current laparoscopic hysterectomy procedures use a device called a morcellator to cut the uterus into small pieces. U.S. Pat. No. 5,569,284 describes a morcellator that employs an auger that can be buried within an organ to process the tissue. The tissue fragments are then carried through the stem of the auger and out of the patient. U.S. Pat. No. 6,997,926 details a tissue morcellator that makes use of a rotating resistance heated electrode to comminute undesirable tissue. Other morcellators use two concentric hollow tubes where a leading edge of the inner tube serves as a blade to cut through tissue that is grasped by forceps and pulled through its hollow core. The process is slow and fatigue-inducing as the surgeon must make precise and repetitive cuts. In addition, the exposed blade of the morcellator runs the risk of causing accidental nicks, resulting in damage that requires open surgery to repair. The coring action can produce small tissue fragments that must be painstakingly removed from the abdominal cavity. Accidental retention of tissue can lead to severe complications.

Accordingly, there exists a need for more efficient and effective methods and devices for macerating and removing tissue in a minimally invasive surgical procedure.

### SUMMARY OF THE INVENTION

The present invention generally provides methods and devices for macerating and removing tissue. In one aspect, a maceration device is provided that includes an elongate hollow member that can be at least partially introduced into a body in a minimally invasive surgical procedure and that has a solid cutting element positioned on its side. A longitudinal axis of the cutting element is substantially parallel to an elongate axis of the elongate hollow member when the elongate hollow member and the cutting element are introduced into a body. The cutting element can rotate to macerate tissue.

The cutting element can have a variety of shapes, sizes, and configurations. For example, the cutting element can be substantially flat. The cutting element can be positioned proximal to a distal end of the elongate hollow member. In some embodiments, the side of the elongate hollow member can include a recess that can seat the cutting element therein. For another example, a rotational plane of the cutting element and a plane parallel to a cross section of the elongate hollow member can be substantially non-parallel. For still another example, a length of the cutting element along the cutting element's longitudinal axis can be larger than a largest cross-sectional dimension of a distal end of the elongate hollow member. In some embodiments, the largest cross-sectional dimension of the distal end of the elongate hollow member can be less than about 1 inch. The cutting element can macerate tissue at any rate, e.g., at a rate greater than about 40 grams per minute.

The maceration device can include a shaft coupled with the elongate hollow member that can deliver power to the cutting element to allow the cutting element to rotate. The shaft can be rotatably disposed within the elongate hollow member, while in some embodiments the shaft can be detachedly coupled to the elongate hollow member.

In some embodiments, the maceration device can also include a tissue containment member that can contain tissue macerated by the cutting element and that can enclose the cutting element and at least a distal end of the elongate hollow member when the cutting element and the distal end of the elongate hollow member are disposed in a body. The tissue containment member can contain a liquid and a gas therein at least at a time the cutting element macerates tissue. The tissue containment member can prevent tissue macerated by the cutting element from coming into contact with an environment within a body and outside the tissue containment member. The tissue containment member can have a variety of shapes, sizes, and configurations. For example, the tissue containment member can be inflatable around the cutting element and at least the distal end of the elongate hollow member. For another example, the tissue containment member can be a deformable bag. In some embodiments, the bag can include an inner layer and an outer layer with a mesh layer disposed between the inner and outer layers. The mesh layer can be pliable when the bag is in an uninflated position and can be rigid when the bag is in an inflated position enclosing the cutting element and at least the distal end of the elongate hollow member. For another example, the tissue containment member can include at least one wire extending along a surface of the tissue containment member that is in electronic communication with a motor providing power to rotate the cutting element. At least partially cutting any one or more wires can stop the motor from providing power.

The maceration device can optionally include a rigid guard member. The rigid guard member can at least partially enclose the cutting element when the cutting element rotates. The rigid guard member can have a variety of shapes, sizes, and configurations. For example, the rigid guard member can include at least two movable arms coupled to the elongate hollow member that can be in a closed position substantially flush with the elongate hollow member when the elongate hollow member is introduced into a body and that can move to an open position extending out from the elongate hollow body to at least partially enclose the cutting element when the cutting element rotates. For another example, the rigid guard member can include a band of synthetic fiber material disposed under the cutting element where a largest diameter of the band of synthetic fiber material is at least as long as a longitudinal length of the cutting element.

In another aspect, a maceration device is provided that includes an elongate member that has a bore therein and that can be disposed in a body. The device also includes a shaft that can rotate while coupled to the elongate member and a substantially flat cutting element coupled to a surface of the elongate member proximal to a distal end of the elongate member. The cutting element can be disposed in a body and rotate to macerate tissue with power provided by the shaft when the shaft rotates. A longitudinal axis of the cutting element and an elongate axis of the elongate member can be substantially non-parallel during at least a portion of the cutting element's rotation. In some embodiments, the shaft is removably coupled to the elongate member.

In yet another aspect, a maceration device is provided that includes a rigid elongate member that can be at least partially introduced into a body through an opening having a largest diameter less than about 2 cm and a rigid cutting element having a longitudinal length greater than about 2 cm and that is coupled to the elongate member proximal to a distal end of the elongate member. The cutting element can be introduced into the body through the opening when the elongate member is being at least partially introduced into the body and can rotate to macerate tissue such that a longitudinal axis of the cutting element is not parallel to an elongate axis of the elongate member during at least a portion of the cutting element's rotation. In some embodiments, the device can also include a motor coupled to the elongate member that can provide power to the cutting element to allow the cutting element to macerate tissue at a rate of about 50 grams per minute to about 500 grams per minute.

### BRIEF DESCRIPTION OF THE DRAWINGS

The invention will be more fully understood from the following detailed description taken in conjunction with the accompanying drawings (not necessarily drawn to scale), in which:

FIG. 1 is a side view of a maceration device;

FIG. 2 is a schematic top view of a cutting element having two teardrop-shaped blades;

FIG. 3 is a schematic top view of a cutting element having two half-ovular-shaped blades;

FIG. 4 is a schematic top view of a cutting element having two irregularly-shaped blades;

FIG. 5 is a schematic top view of a cutting element having two substantially triangular-shaped blades;

FIG. 6 is a schematic top view of a cutting element having two curved or substantially C-shaped blades;

FIG. 7 is a schematic top view of a cutting element having a single diamond-shaped blade;

FIG. 8 is a perspective view of a substantially cylindrical cutting element;

FIG. 9 is a schematic top view of the maceration device of FIG. 1;

FIG. 10 is a schematic view facing a distal end of the maceration device of FIG. 1;

FIG. 11 is a schematic side view of a maceration device having a recess formed therein for seating a cutting element;

FIG. 12 is a schematic view facing a distal end of the maceration device of FIG. 11;

FIG. 13 is a side view of a distal portion of the maceration device of FIG. 1 having its cutting element at least partially removed;

FIG. 14 is a side view of the cutting element of FIG. 13;

FIG. 15 is a side view of a cutting element being coupled to the maceration device of FIG. 1;

FIG. 16 is a schematic cross-sectional view of a maceration device;

FIG. 17 is a cross-sectional view of a handle of a maceration device;

FIG. 18 is a schematic side view of a maceration device having a belt drive power system;

FIG. 19 is a schematic side view of a maceration device having a geared power system;

FIG. 20 is a schematic cross-sectional side view of two ports that can be coupled to form a maceration device;

FIG. 21 is a schematic side view of the ports of FIG. 20 coupled together to form a maceration device;

FIG. 22 is a schematic side view of a maceration device having a cutting element with a protective band coupled thereto;

FIG. 23 is a schematic view of a maceration device having a tissue containment member and a guard member coupled thereto;

FIG. 24 is a schematic side view of a tissue containment member having wires coupled thereto;

FIG. 25 is a schematic cross-sectional view of a tissue containment member having a two pliable bag layers separated by and coupled together with a protective layer;

FIG. 26 is a side view of a maceration device having a tissue containment member coupled thereto and in an unexpanded position;

FIG. 27 is a side view of the maceration device of FIG. 26 with the tissue containment member in an expanded position;

FIG. 28 is a side view of the maceration device of FIG. 27 with tissue disposed in the tissue containment member;

FIG. 29 is a side view of the maceration device of FIG. 28 macerating tissue in the tissue containment member;

FIG. 30 is a side view of the maceration device of FIG. 29 with the tissue containment member substantially free of tissue; and

FIG. 31 is a side view of a maceration device having a guard member coupled thereto that contains tissue.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention generally provides methods and devices for macerating and removing tissue. While the methods and devices disclosed herein can be used in conventional, open surgical procedures, they are particularly useful in minimally invasive surgical procedures, particularly laparoscopic surgery and endoscopic procedures. The principles described herein can be applicable to the particular types of tools described herein and to a variety of other surgical tools having similar functions. In addition, the tools can be used alone in a surgical procedure, or they can be used in conjunction with other devices that facilitate minimally invasive surgical procedures. A person skilled in the art will appreciate that the present invention has application in conventional endoscopic and open surgical instrumentation as well application in robotic-assisted surgery. While a surgical device can be introduced to a body in any way and used to macerate any tissue for any purpose, in an exemplary embodiment the surgical device is configured for introduction into a body through a man-made orifice and for use in macerating and removing tissue, e.g., an unhealthy organ (e.g., a uterus, a kidney, etc.), a tissue growth, malignant tissue, fibroids, abdominal masses, and other undesirable tissue.

Some embodiments are drawn to a surgical device that can macerate tissue and remove tissue from a body. In an exemplary embodiment, the surgical device includes a morcellator that can be distally advanced into a body in a minimally invasive surgical procedure and positioned proximate to tissue desirable for removal from the body. The morcellator can include an elongate shaft having a cutting element positioned on the shaft's side (i.e., not located on a distal tip of the elongate shaft). The cutting element can rotate to macerate tissue. When being introduced to the body, an elongate axis of the elongate shaft and a longitudinal axis of the cutting element can be substantially parallel to each other. When the cutting element rotates, the elongate axis of the elongate shaft and longitudinal axis of the cutting element can not be parallel during at least a portion of the cutting element's rotation. In this way, the cutting element can be introduced to a body through a minimally invasive surgical opening (e.g., an incision or other orifice having a length of less than about one inch) while having a longitudinal length larger than a maximum diameter of the opening used to introduce the morcellator including the cutting element into a body. The cutting element can thus rotate through a cutting surface having a maximum diameter equal to the cutting element's longitudinal length rather than a smaller cutting surface having a maximum diameter no greater than the surgical opening's length, thereby increasing the amount of tissue within the cutting element's rotational reach. Being able to reach more tissue, the morcellator can macerate tissue more quickly and reduce an amount of time necessary to perform the surgical procedure. Processing tissue more quickly can reduce expense of surgery and reduce physician fatigue. Furthermore, the morcellator can include a containment member configured to contain tissue macerated by the cutting element, thereby protecting surrounding tissue from accidental cutting or other damage by the cutting element that can require further surgical time, if not a more invasive open surgical procedure, to repair. A guard member coupled to the morcellator and at least partially surrounding the cutting element can also help protect surrounding tissue from the cutting element. The containment member can also help contain cut tissue and prevent dispersal of cut tissue in the body, thereby preventing cut tissue from dispersing in the body, requiring time to locate and retrieve, and from remaining within the body and potentially causing severe complications, particularly if the macerated tissue includes malignant tissue.

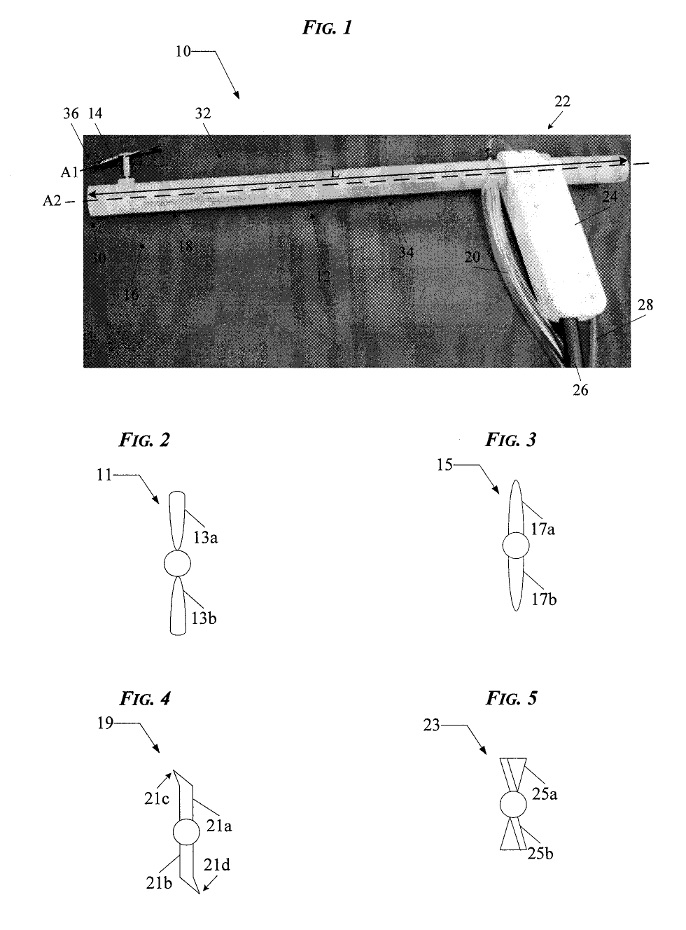

The morcellator can have a variety of configurations. In an exemplary embodiment shown in FIG. 1, a morcellator 10 can include an elongate member, e.g., a shaft 12, having a cutting element, e.g., a knife or blade 14, coupled to the shaft 12 in the shaft's distal portion 16. A surgeon or other medical professional can hold the morcellator 10 by a handle 24 coupled to the shaft 12 in the shaft's proximal portion 22 and guide the blade 14 in position proximate to tissue to be macerated. A power cable 26 can be coupled to the shaft 12 at the shaft's proximal portion 22 and provide power to the morcellator 10, e.g., using a high speed motor at the cable's proximal end (not shown). Power from the cable 26 can drive rotation of the blade 14. While the blade 14 rotates, a fluid tube 28 at the shaft's proximal portion 22 can provide a fluid (liquid and/or gas) that can flow through a hollow interior of the shaft 12 and out of the shaft 12 at the shaft's distal portion 16. The shaft's distal portion 16 can also include aspiration holes 18 through which tissue cut by the blade 14 and/or fluid can be aspirated into the hollow interior of the shaft 12. Aspirated tissue can travel through a hollowed portion of the shaft 12 and out a suction tube 20 at the shaft's proximal portion 22. The suction tube 20 can also provide suction to help draw tissue and/or fluid into the aspiration holes 18, e.g., using a suction pump at the suction tube's proximal end (not shown).

The morcellator 10 can be formed from a variety of materials but is preferably formed from any combination of one or more biocompatible materials safe for use in the body. While the morcellator 10 can be formed from any combination of rigid or flexible materials, the various components of the morcellator 10 are preferably rigid, except as discussed herein. For example, the power cable 26, the fluid tube 28, and the suction tube 20 can be at least partially made from a flexible material.

The morcellator 10 can have any size, shape, and configuration, as will be appreciated by a person skilled in the art. The morcellator 10 preferably has a size in at least the shaft's distal portion 16 that allows use of the morcellator 10 in a minimally invasive surgical procedure. As such, the shaft's distal portion 16 preferably has a maximum cross-sectional dimension less than about one inch, and more preferably less than about 1.5 cm or less than about 0.5 cm, to allow insertion of at least part of the shaft's distal portion 16 through a small opening in a body. The shaft's size and shape can be the same or can vary along its longitudinal length L.

The morcellator's blade 14 can also have any shape, size, and configuration, but the blade 14 is preferably configured to macerate tissue. The blade 14 is also preferably configured to have a size that allows its insertion into a body in a minimally invasive surgical procedure by having a maximum width equal to or less than a maximum diameter of a surgical opening, e.g., less than about one inch and more preferably less than about 1.5 cm or less than about 0.5 cm. As mentioned above, the blade's maximum longitudinal length, which can have any length, e.g., about 3 cm to about 5 cm, can be larger than its maximum width which can allow the blade 14 to have a larger surface plane of rotation.

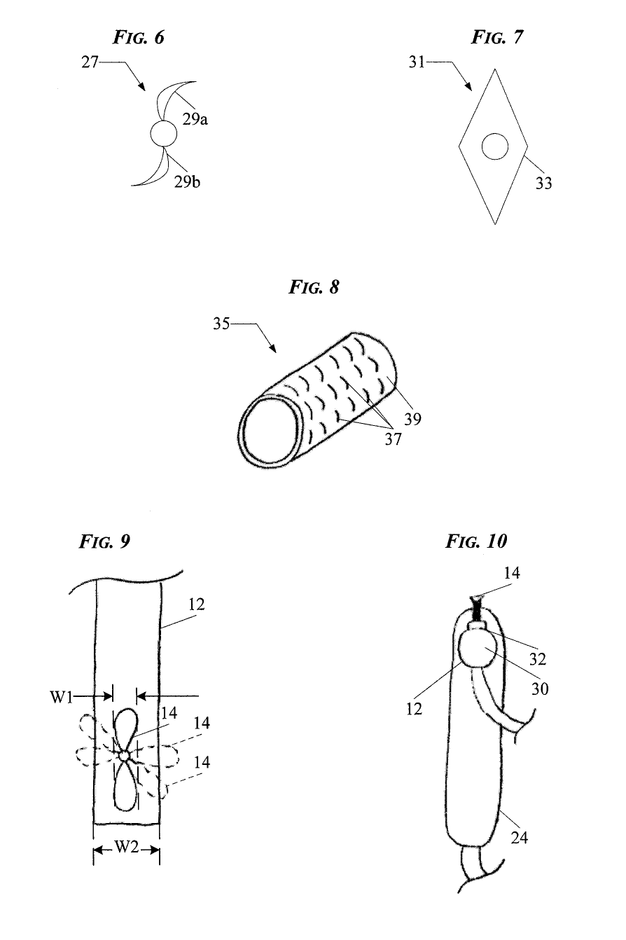

While the blade 14 is shown in FIG. 1 as a single blade, the blade 14 can include two or more individual blades that can be coupled to and rotate around a center rod or shaft. Moreover, the blade 14 can be substantially planar, angular, or movable between planar and/or angular positions, which can help orient the blade 14 during introduction to or withdrawal from a body. If the blade 14 has a right-angled configuration, gravity can help push tissue into the blade 14. By way of non-limiting example, FIGS. 2-8 illustrate various embodiments of cutting elements that can be used with a morcellator device described herein. In general, each of the cutting elements 11, 15, 19, 23, 27, 31 includes one or more individual blades having a particular shape, same or different from other blades on the same cutting element, such as a rectangular shape, a curved shape, a triangular shape, a square shape, or an irregular shape. Blades on cutting elements including more than one blade can be equidistantly or otherwise spaced. FIG. 2 illustrates a cutting element 11 having two teardrop-shaped blades 13a, 13b. FIG. 3 illustrates a cutting element 15 having two half-ovular-shaped blades 17a, 17b. FIG. 4 illustrates a cutting element 19 having two irregularly-shaped blades 21a, 21b having pointed tips 21c, 21d. FIG. 5 illustrates a cutting element 23 having two substantially triangular-shaped blades 25a, 25b. FIG. 6 illustrates a cutting element 27 having two curved or substantially C-shaped blades 29a, 29b. FIG. 7 illustrates a cutting element 31 having a single diamond-shaped blade 33.

FIG. 8 illustrates a substantially cylindrical cutting element 35 having a plurality of blade elements 37 on its surface 39. The cutting element 35 can be disposed around the shaft 12, integrally formed with the shaft 12, disposed in a housing coupled to the shaft 12, or otherwise coupled to the shaft 12. The cutting element 35 can be recessed in the shaft 12 or can extend any distance from the shaft 12 at any angle. A tissue containment member and/or a rigid guard member, discussed further below, can each be configured to enclose the cutting element 35. Tissue can be directed against the cutting element 35, for example, by withdrawing a tissue containment member containing tissue to be macerated toward the cutting element 35, by placing tissue within a guard member proximate to the cutting element 35, or by having a fluid irrigation sucked through the cutting element 35 while the cutting element 35 is spinning to create a vacuum force. The fluid inflow can come from a second port or from a different channel on the same port.

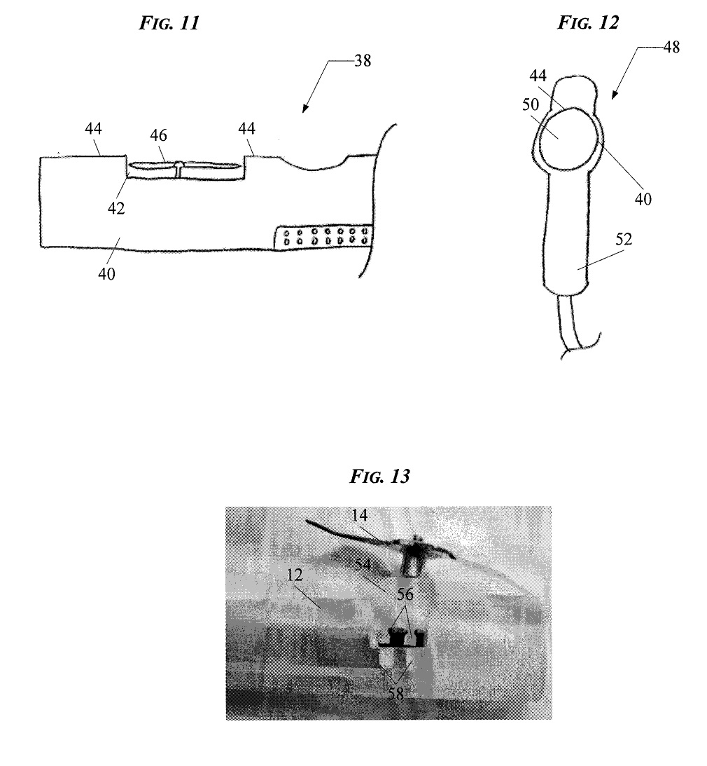

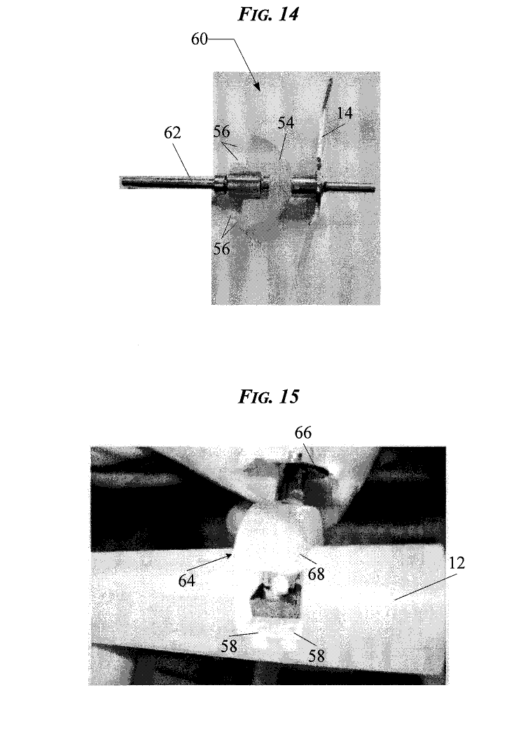

Referring again to FIG. 1, the blade 14 can be located anywhere on the shaft 12, but as mentioned above, the blade 14 is preferably coupled to the shaft 12 in the shaft's distal portion 16 to help minimize a length of the shaft 12 disposed in a body to macerate tissue using the morcellator 10. Although the blade 14 is shown disposed on a top surface 32 of the shaft 12, e.g., a surface opposite a bottom surface 34 from which the handle 24 generally extends, the blade 14 can be disposed on any surface of the shaft 12. In other words, the plane of rotation of the blade 14 can not be parallel to a cross sectional plane of the shaft 12. The blade 14 is also preferably coupled to the shaft 12 proximate to a distal tip 30 of the shaft 12, e.g., any length proximally beyond the shaft's distal tip 30 along the shaft's longitudinal length L. In other words, the morcellator's operative surface can be on the morcellator's side rather than on its distal tip 30. In this way, when the morcellator 10 is distally advanced into a body, the shaft's distal tip 30 can “lead” the morcellator 10 rather than the blade 14. Correspondingly, the blade 14 is preferably sized such that at least when a longitudinal axis A1 of the blade 14 is substantially parallel to an elongate axis A2 of the shaft 12, e.g., when the blade 14 is in a non-rotating position (e.g., when the morcellator 10 is being introduced or withdrawn from a body), a distal end 36 of the blade 14 does not extend beyond the shaft's distal tip 30. As shown in FIG. 9, a maximum width W1 of the blade 14 is preferably less than or equal to a maximum cross-sectional width W2 of the shaft 12 in at least in the shaft's distal portion 16 such that the blade 14 does not extend beyond the maximum cross-sectional width W2 of the shaft 12 to help allow the shaft 12 rather than the blade 14 to come into contact with tissue or other material when the morcellator 10 is being introduced into or withdrawn from a body. However, as shown by the blades 14 in shadow in FIG. 9, during at least a portion of the blade's rotation, which can be in a clockwise or a counterclockwise direction, the blade 14 can extend beyond the maximum cross-sectional width W2 of the shaft 12, thereby allowing the blade 14 to access a greater amount of tissue and macerate tissue more quickly than if limited in size to the cross-sectional width W2 of the shaft 12. Also as shown in FIG. 9, during at least a portion of the blade's rotation, the blade's longitudinal axis can be orthogonal to the shaft's elongate axis.

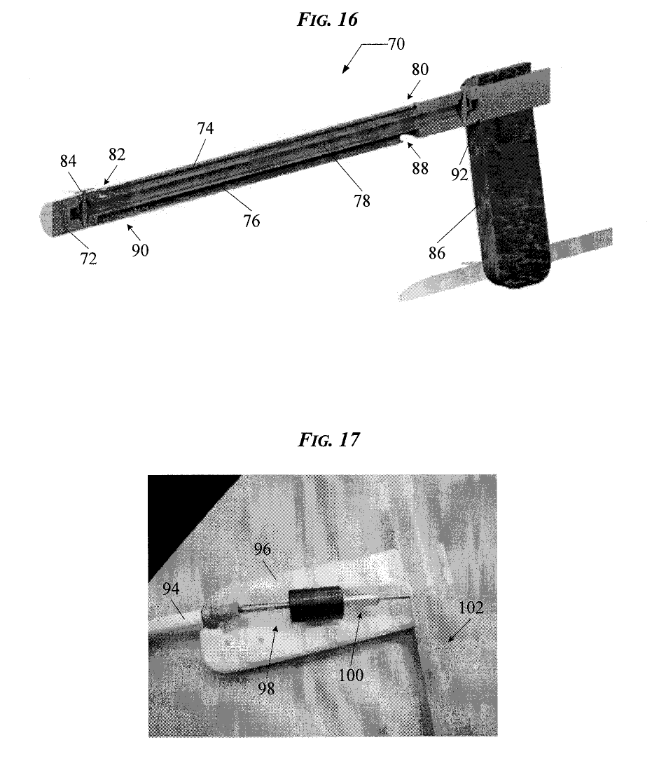

As shown in a view directly facing a distal end 50 of the morcellator 10 in FIG. 10, the blade 14 can extend a distance beyond the shaft's top surface 32. Alternatively, the blade 14 can be substantially flush with, e.g., sit or rest upon, or be recessed in the shaft's top surface 32 (or whatever surface the blade 14 is coupled to). For example, as illustrated in FIG. 11, a distal portion 38 of a morcellator shaft 40 can include a recess 42 in its surface 44 that is configured to seat a cutting element 46. The recess 42 can have any shape and size, but the recess 42 preferably has a length at least long enough to seat the cutting element 46 in a non-rotating position, e.g., when elongate axes of the shaft 40 and the cutting element 46 are substantially parallel. The recess 42 also preferably extends widthwise through the shaft's surface 44 such that the shaft 40 does not interfere with the cutting element's rotation. The recess 42 can fully seat the cutting element 46 such that the cutting element 46 does not extend beyond the shaft's surface 44, as shown in a distal-end view of a morcellator 48 in FIG. 12 where the cutting element 46 and the recess 42 are not visible beyond the distal end 50 of the shaft 40, but any or all of the cutting element 46 can extend any distance beyond the shaft's surface 44.

The cutting element 46 can be configured to be movable in any one or more directions within the recess 42 such that the cutting element 46 can change its positioning within and/or outside the recess 42. In this way, the cutting element 46 can be introduced into a body in a non-rotating position while seated in the recess 42 and can move at least partially outside the recess 42 to potentially have better access to tissue when rotating and cutting tissue. The morcellator's handle 52 can include controls for actuating movement of the cutting element 46.

Referring again to FIG. 1, the blade 14 can be fixedly or removably coupled to the shaft 12. If the blade 14 is removably coupled to the shaft 12, the blade 14 can be removed from the shaft 12 and replaced with another blade coupled to the shaft 12, or the blade 14 can be re-coupled to the shaft 12 after cleaning, sharpening, inspecting, or otherwise processing the blade 14. A person skilled in the art will appreciate that the blade 14 can be removably coupled to the shaft 12 in a variety of ways. As shown in FIG. 13 by way of non-limiting example only, the blade 14 can be coupled to a coupling element 54 including one or more male mating elements 56 corresponding to one or more female mating elements 58 in the shaft 12, although the blade's mating elements can be female and correspond to male shaft mating elements. The blade's and shaft's mating elements 56, 58 can mate together to lock the blade 14 to the shaft 12, but the mating elements 56, 58 can be snapped apart or otherwise de-coupled to release the blade 14 from the shaft 12. A blade construction 60, shown in FIG. 14, including the blade 14 and the coupling element 54 can be removed from the shaft 12. The blade construction 60 can also include a center rod or shaft 62 that can be a center axis around which the blade 14 can rotate and that can be used to help provide power to rotate the blade 14 as further discussed below. Following removal of the blade construction 60 from the shaft 12, another blade construction 64 can be coupled to the shaft 12, as shown in FIG. 15. The other blade construction 64, which is a non-limiting example only, includes a generally elliptical blade 66 coupled to a coupling element 68 that can mate with the shaft 12 via the shaft's mating elements 58.

As mentioned above, a morcellator can include an elongate member having at least one hollow portion or bore included therein. FIG. 16 illustrates a morcellator 70 including an elongate shaft 72 having a fluid channel 74, an aspiration channel 76, and a drive shaft 78 extending within the shaft's longitudinal length. As will be appreciated by a person skilled in the art, the fluid channel 74, the aspiration channel 76, and the drive shaft 78 can each have a variety of configurations, include one or more separate channels therein, and can be combined in any way, although preferably none are in communication with each other. The fluid channel 74, for example, can include one or more separate channels and can provide one or more individual fluids. Furthermore, fluid and suction can be applied in a variety of other ways, with or without using the channel(s) 74, 76, as will be appreciated by a person skilled in the art. By way of non-limiting example only, a guard member coupled to the morcellator 70 can provide fluid to the system.

One or more of the fluid channel 74, the aspiration channel 76, and/or any other supply channels can include a pressure sensing mechanism coupled or otherwise in communication therewith to detect if a pressure in a channel rises above a threshold level, preferably a pre-programmed level specified by a physician or other medical professional, which can be the same or different for different channels. If the pressure level is exceeded in a certain channel, one or more valves can be switched to aspirate such that fluid can be aspirated. In this way, clogs can be detected and addressed.

The fluid channel 74 can have a proximal opening 80 configured to couple to a fluid source via a fluid tube, where the fluid can be driven by a pump. Fluid can flow from the proximal opening 80, through the fluid channel 74, and out a distal opening 82 configured to allow fluid release into an external environment, e.g., proximate to tissue to be macerated by a blade 84. The presence of fluid, preferably a combination in any ratio of a liquid and a gas, in the external environment can aid the blade 84 in cutting tissue by helping to promote tissue flow. The fluid channel's proximal and distal openings 80, 82 can be located anywhere along the shaft 72 and/or a handle 86 of the morcellator 70, but the proximal and distal openings 80, 82 are preferably proximal to the blade 84 to help avoid interfering with the blade 84 and/or its power supply.

The aspiration channel 76 can also have a proximal opening 88 and a distal opening 90. The aspiration channel's proximal opening 88 can be configured to couple to a suction source, e.g., a vacuum pump, via a suction tube. Material, e.g., tissue, fluid, etc., proximate to the distal opening 88 can be pulled or suctioned into the aspiration channel 76 by the force provided by the suction source, pass through the aspiration channel 76, and exit the shaft 72 and/or the handle 86 through the aspiration channel's proximal opening 88. The distal opening 90 can include one or more openings, such as a mesh of aspiration holes configured to act as a filter to help ensure that only small pieces of material can pass into the aspiration channel 76, which can reduce blockage of the aspiration channel 76, and be removed from a body through a minimally invasive surgical opening. The aspiration channel's proximal and distal openings 88, 90 can be located anywhere along the shaft 72 and/or the handle 86, but the proximal and distal openings 88, 90 are preferably proximal to the blade 84 to help avoid interfering with the blade 84 and/or its power supply. Irrigation via the fluid channel 74 and suction via the aspiration channel 76 can occur simultaneously to help provide a rapid, continuous tissue maceration process.

Generally, the drive shaft 78 can house a drive mechanism configured to rotate the blade 84. A power source, e.g., a high speed motor, can be coupled to the drive mechanism disposed in the drive shaft 78 at a proximal end 92 of the drive shaft 78, such as by a drive cable (not shown). Any amount of power can be delivered to the blade 84 via the drive shaft 78.

Sufficient power can be provided via the drive shaft 78, in some embodiments, to macerate a large amount of tissue in a short amount of time and in a shorter amount of time than in prior art morcellators. Even while allowing the blade 84 to be introduced into a body in a minimally invasive surgical procedure, enough power can be delivered to allow maceration of tissue by the blade 84 at a rate, by ways of non-limiting example only, greater than about twelve grams per minute, greater than about forty grams per minute, in a range from about fifty grams per minute to about three hundred grams per minute, and in a range from about fifty grams per minute to about five hundred grams per minute. At a rate greater than about 40 g/min, a tissue about the size of a typically sized uterus can be macerated less than about one minute, compared to about 20-30 minutes for prior art morcellators having rates of about 5 g/min to about 12 g/min.

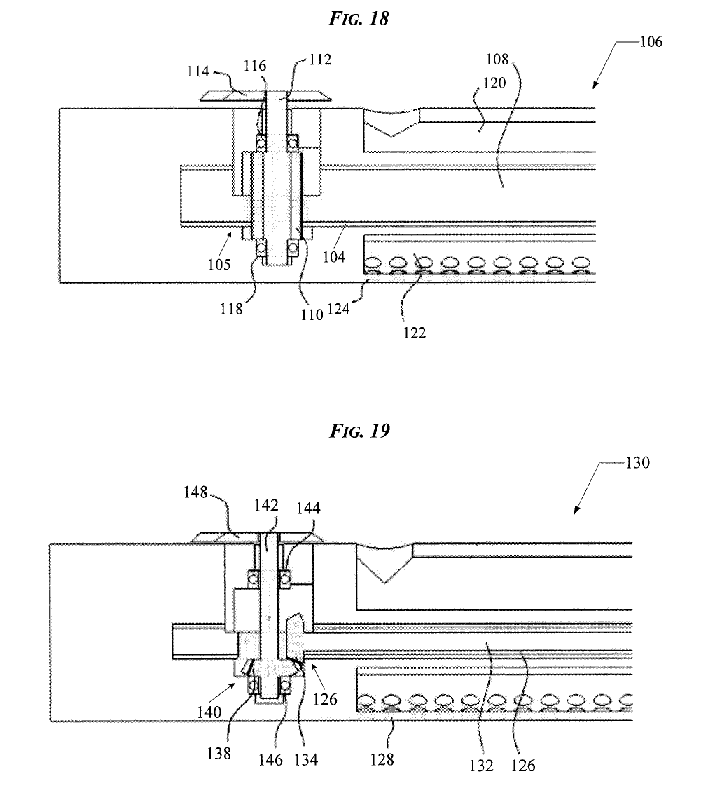

By way of non-limiting example only, FIG. 17 shows a drive cable 94 extending from outside a morcellator handle 96 and into a hollowed portion 98 of the handle 96 with the drive cable 94 coupled to a drive mechanism 100 extending at a substantially right angle into a drive shaft 102. As will be appreciated by a person skilled in the art, the drive mechanism housed in the drive shaft can have a variety of configurations. In one embodiment, shown in FIG. 18, a drive mechanism housed in a drive shaft 104 within an elongate shaft 124 of a morcellator 106 can include a belt drive. The belt drive can include a toothed belt 108 coupled to two distal spindles 110, or any number of spindles, in a distal portion 105 of the drive shaft 104. Power can be input to the belt drive by rotating one or more spindles at a proximal end (not shown) of the drive shaft 104, which via the belt 108 can cause rotation of the distal spindles 110, which can rotate a cutting element rod or shaft 112 coupled to a cutting element 114. Upper and lower bearings 116, 118 can help support the cutting element rod 112 to help increase efficiency of the belt drive. The morcellator 106 can also include a fluid channel 120 and an aspiration channel 122 as discussed above.

In another embodiment, the drive mechanism can include a hydraulic or pneumatic spindle, e.g., a small, high speed shaft similar to what can be used in dental drilling equipment. The hydraulic or pneumatic spindle is similar to the belt drive discussed above, but the toothed belt preferably has wider teeth, resembling a paddle wheel. High pressure, high velocity fluid can stream through the morcellator's drive shaft, causing high speed rotation of a rod or shaft coupled to a cutting element.

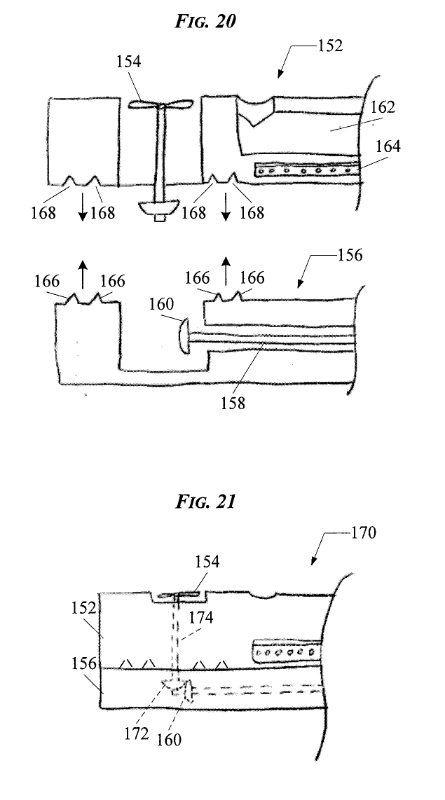

In yet another embodiment, shown in FIG. 19, a drive mechanism housed in a drive shaft 126 within an elongate shaft 128 of a morcellator 130 can include a geared mechanism. The geared mechanism can include a drive axle 132 disposed in the drive shaft 126 and having a gear 134, e.g., a miter gear, at its distal end 136. The gear 134 can engage a second gear 138, e.g., a miter gear, at a proximal end 140 of a cutting element rod or shaft 142 supported by upper and lower bearings 144, 146 and having a cutting element 148 at its distal end 150. When a power source coupled to a proximal end (not shown) of the drive axle 132 provides power to rotate the drive axle 132, the drive axle's gear 134 also rotates, thereby causing the second gear 138 and hence the cutting element rod 142 and the cutting element 148 to rotate.

A morcellator can optionally include multiple separate instruments configured to couple together to form the morcellator. Generally, one instrument can include a cutting element, another instrument can include a power supply, and the two instruments can be assembled together inside or outside a body to form a morcellator. In this way, the morcellator can have a less complicated internal design such that if any functionality of the morcellator breaks or needs maintenance or replacement, only the instrument including that broken or malfunctioning aspect can be affected. Having fewer elements, that aspect can be easier to repair than a single-instrument morcellator. Furthermore, the other instrument(s) of the morcellator can continue to be used with other, functional instrument(s).

As shown in one embodiment of a multi-port morcellator in FIG. 20, a first instrument 152 can include a cutting element 154 while a second instrument 156 configured to mate with the first instrument 152 can include a power source, illustrated here as a geared mechanism including a drive axle 158 and a drive gear 160. The first instrument 152 also includes a fluid channel 162 and an aspiration channel 164, but either instrument 152, 156 can include one or both of the fluid and aspiration channels 162, 164. Other morcellator elements, such as a guard member (not shown), a containment member (not shown), and/or any other elements, can be included as part of either instrument 152, 156. As will be appreciated by a person skilled in the art, the first and second instruments 152, 156 can be mated together in a variety of ways, such as by pushing or snapping one or more protrusions 166 in one of the instruments, here the second instrument 156, into corresponding depressions 168 in the other instrument, here the first instrument 152. Mating the first and second instruments 152, 156 together, as shown in FIG. 21, can form a morcellator 170 with the drive gear 160 engaging a cutting element gear 172 coupled to the cutting element 154 via a cutting element rod 174.

As mentioned above, a guard member can optionally be coupled to a morcellator and be configured to help prevent the morcellator's cutting element from accidentally cutting or otherwise damaging tissue not intended for maceration by the cutting element. The guard member can also help stabilize tissue during cutting by the morcellator's cutting element. Generally, the guard member can at least partially enclose the cutting element at least when the cutting element is rotating. The guard member can have any size, shape, and configuration and can be rigid and/or flexible, although the guard member is preferably rigid.

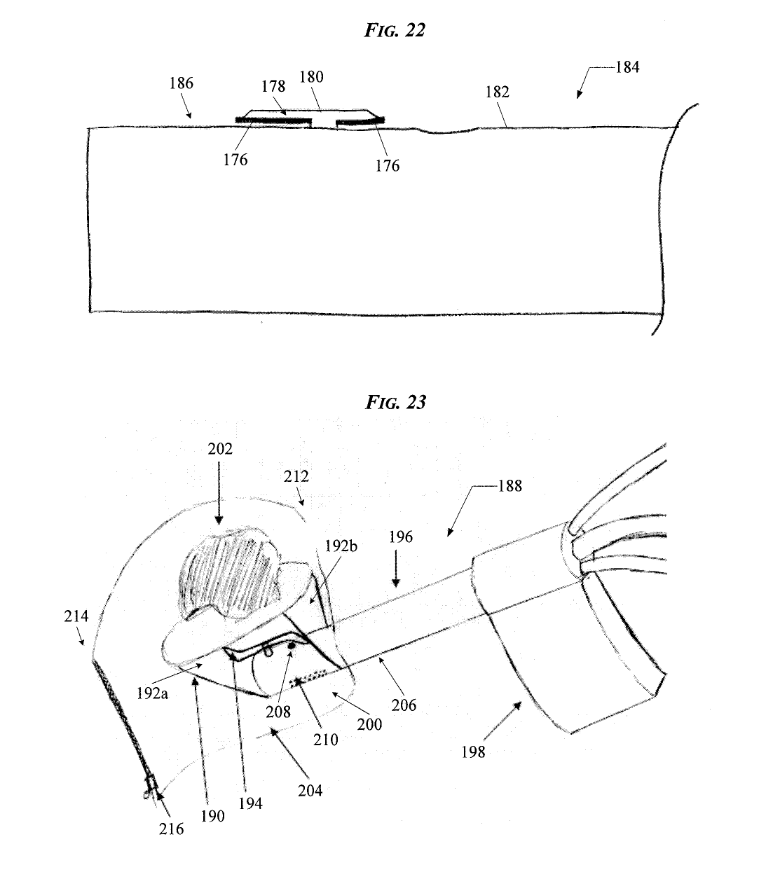

FIG. 22 illustrates one embodiment of a guard member coupled to a morcellator 184, a band 176 of synthetic fiber material disposed on a distal surface 178 of a cutting element 180, e.g., a surface substantially facing a surface 186 of an elongate member 182 to which the cutting element 180 is coupled. The synthetic fiber material can have a variety of compositions, such as a para-aramid fiber, e.g., Kevlar™ manufactured by DuPont of Wilmington, Del., configured to be cut-resistant and preferably biocompatible. The band 176 can have any size, shape, and configuration, but the band 176 preferably has an area at least as large as the cutting element 180 to help ensure that the band 176 covers the cutting element's distal surface 178. The band 176 can extend any distance beyond the cutting element's edges and can extend at any angle(s) from the cutting element 180. In this way, the band 176 can help prevent the cutting element 180 from cutting any tissue or other material slipping toward, sliding near, or otherwise approaching the cutting element 180 other than tissue intentionally positioned adjacent to the cutting element 180 above its distal surface 178.

FIG. 23 illustrates another embodiment of a guard member coupled to a morcellator 188, a collapsible cup 190 formed from a plurality of movable arms 192a, 192b. Although the cup 190 includes two arms 192a, 192b, the cup 190 can include any number of movable arms. The arms 192a, 192b can have any size, shape, and configuration and can be made from any material, preferably a biocompatible, cut-resistant material. The arms 192a, 192b can be fixedly or removably coupled to the morcellator 188. The arms 192a, 192b can move between at least two positions. The arms 192a, 192b can have a closed position where the arms 192a, 192b can be substantially flush with an elongate shaft 196 of the morcellator 188 or at least partially disposed within the shaft 196 and/or a recess formed in the shaft 196 such that the arms 192a, 192b do not increase the shaft's cross-sectional dimension or increase the shaft's cross-sectional dimension to an extent still allowing at least a distal portion 200 of the morcellator 188 to be introduced into a body in a minimally invasive surgical procedure. The arms 192a, 192b can also have an open position, as shown, where the arms 192a, 192b extend at any angle(s) from the shaft 196 to form the cup 190 such that the arms 192a, 192b at least partially enclose a cutting element 194 coupled to the morcellator's shaft 196. The arms 192a, 192b preferably fit together to form a substantially closed surface, e.g., a substantially fluid tight seal, at least partially surrounding the cutting element 194. The cup 190 preferably includes at least one open portion to allow fluid exiting the shaft 196 from a fluid outlet 208 of a fluid channel to access the cutting element 194 and to allow fluid and macerated pieces of a tissue 202 to access aspiration holes 210 and be drawn into the shaft 196. The arms 192a, 192b preferably extend at least from a bottom-most position of the cutting element 194 in a rotating position to a top-most position of the cutting element 194 in a rotating position. More preferably, the arms 192, 192b extend from a bottom surface 206 of the shaft 196 to at least the top-most position of the cutting element 194 in a rotating position such that any tissue or other material not intended for maceration that approaches the cutting element 194 in the morcellator's distal portion 200, e.g., a containment member 204 or tissue disposed outside the containment member 204, can be prevented from encountering the cutting element 194 by the arms 192a, 192b. The arms 192a, 192b can be movable between the open and closed positions, for example, via actuating controls at the morcellator's handle 198. Preferably, the arms 192a, 192b are moved from the closed position to the open position prior to the cutting element 194 rotating and macerating tissue 202 disposed within the containment member 204, as discussed further below.

The containment member 204 as illustrated in FIG. 23 is a pliable or deformable bag, but the containment member 204 can have a variety of configurations. The containment member 204 can have any size, shape, and configuration and can be formed from any combination of, preferably flexible and biocompatible, materials, e.g., a plastic, a polymer, a flexible metal such as spring steel, a shape memory material such as a nickel-titanium alloy (e.g., Nitinol), a copper-zinc-aluminum-nickel alloy, a copper-aluminum-nickel alloy, a nickel-titanium alloy, and a thermoplastic material such as nylon, and other types of surgically safe materials. While the containment member 204 is illustrated as substantially transparent, the bag can be transparent, translucent, opaque, or any combination thereof. The containment member 204 can be fixedly or removably coupled to the morcellator 188 and is preferably configured to enclose the morcellator's distal portion 200, including the guard member 190, the cutting element 194, the fluid outlet 208, and the aspiration holes 210. A proximal portion 212 of the containment member 204 can be closed and coupled with a substantially fluid tight seal to the shaft 196 in the morcellator's distal portion 200, although the containment member 204 can be coupled to the morcellator 188 in any way appreciated by a person skilled in the art. A distal portion 214 of the containment member 204, or any other portion(s) of the containment member 204, can be configured to have open and closed positions, such as by using a zipper locking seal 216 or any other sealing mechanism as will be appreciated by a person skilled in the art. In the open position, the containment member's distal portion 214 can provide access to an internal cavity of the containment member 204 such that material, e.g., the tissue 202, can be disposed within the containment member 204. The zipper locking seal 216 can be partially or fully open in the open position. In the closed position, the containment member's distal portion 214 can form a substantially fluid tight seal such that any material disposed within the containment member's internal cavity cannot escape easily or at all from the internal cavity through the containment member 204 (the material can exit the containment member 204 in other ways, such as through the aspiration holes 210). The containment member 204 can be configured to inflate with fluid introduced into the containment member's internal cavity such that the containment member 204 has a sufficient volume to help prevent the cutting element 194 from coming into contact with the containment member 204 when the cutting member 204 rotates.

The containment member 204 can optionally include an opening in its proximal portion 212 through which at least the distal portion 200 of the morcellator 188 can be passed. If the morcellator is a multi-port morcellator, then the containment member can include multiple openings to accommodate the multiple ports, e.g., one opening for a shaft including a cutting element and one opening for a shaft including a fluid channel. The morcellator 188 and the containment member 204 as separate elements can be concurrently or sequentially introduced into a body through a minimally invasive surgical opening, and the morcellator 188 can be distally advanced into the containment member's proximal opening. Such a containment member configuration can allow larger and/or more complicated containment members, such as with integral guard members, which would not fit through the minimally invasive surgical opening if introduced simultaneously with the morcellator's shaft 196. Similarly, a guard member can be inserted into a body separately from a containment member and/or a morcellator and coupled to the containment member and/or the morcellator inside the body.

Generally, the containment member 204 can be configured, with the seal 216 in the closed position, to contain the tissue 202 to be macerated by the cutting element 194. In this way, when the cutting element 194 macerates the tissue 202, pieces of the tissue 202 can be prevented from dispersing in an environment outside the containment member 204. Additionally, fluid introduced into the containment member 204 through the fluid outlet 208 can also be contained separate from the outside environment. The containment member 204 can be removed from a body after the tissue 202 has been satisfactorily macerated, so any tissue fragments or other material that does not get suctioned through the aspiration holes 210 and remains in the containment member 204 can be removed from the body along with the containment member 204.

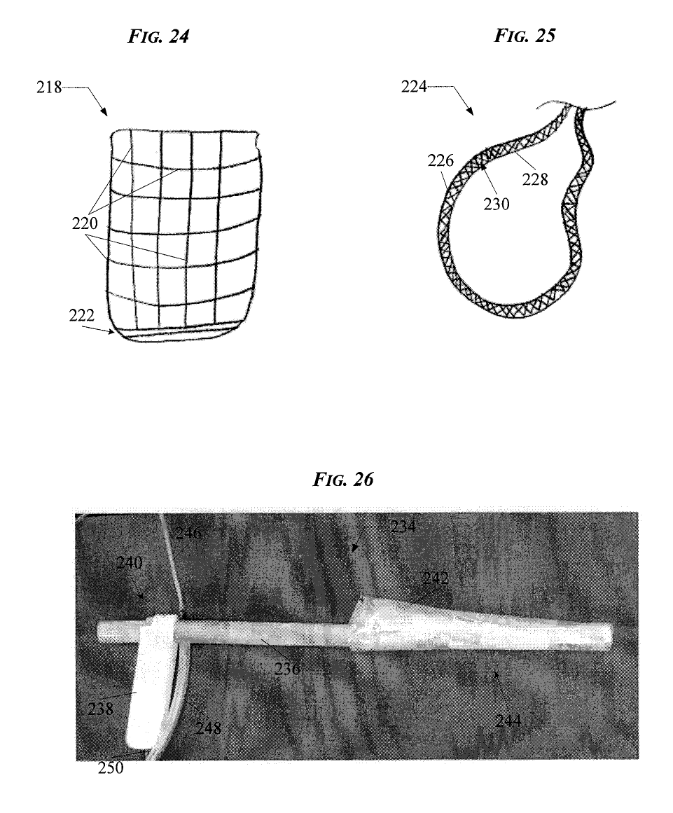

In some embodiments, a containment member can be configured to provide the additional functionality of a guard member. For example, the containment member 204 can include a cut-resistant coating, e.g., a synthetic fiber material, Kevlar™, etc., over at least a portion of its inside and/or outside surfaces. In one embodiment shown in FIG. 24, a containment member 218 can include a pliable bag similar to the containment member 204, but the containment member 218 has a plurality of feedback sensors or wires 220 integrated into, formed on, or otherwise coupled thereto. The wires 220 can be made from any combination of conductive, preferably biocompatible metal, materials. The wires 220 are illustrated as thin strands arranged on the containment member 218 in a checkerboard-style pattern over the containment member's surface, but the wires 220 can have any size, shape, and configuration, including a configuration of one or more feedback sensors. The wires 220 can also have any arrangement in or on the containment member 218, but the wires 220 preferably extend circumferentially around the containment member 218, while allowing a seal 222 to be formed, e.g., by a zipper locking seal, a cinch, etc., such that the bag can have an open position. The wires 220 can be coupled to a power supply providing power to a morcellator's cutting element such that cutting or otherwise severing any one or more of the wires 220 can break the power supply to the cutting element. In other words, cutting at least one of the wires 220 can stop the cutting element from rotating. Cutting or otherwise severing any one or more of the wires 220 can also or instead cease fluid from flowing into the containment member 218 and/or remove application of suction. In this way, if the containment member 218 is torn, sliced, or otherwise punctured, such as by the containment member 218 coming into contact with a spinning cutting element, to disturb a substantially fluid tight seal the containment member 218 forms around a distal portion of a morcellator, the cutting element can cease rotation to help prevent any macerated tissue and/or other material disposed within the containment member 218 from being further circulated and possibly dispersed into an outside environment.

In another embodiment of a containment member combined with a guard member, shown in FIG. 25, a containment member 224 can include a pliable bag similar to the containment member 204 above, but the containment member 224 includes outer and inner pliable bag layers 226, 228 separated by and coupled together with a protective layer 230. Although the containment member 224 includes two bags 226, 228, the containment member 224 can include any number of bags separated by any number of protective layers. The protective layer 230 can have a variety of configurations, but generally, the protective layer 230 includes a fiber or plastic mesh material, e.g., a honeycomb material, configured to have pliable and rigid states. When the containment member 224 is in a collapsed position, such as when being introduced into a body, the protective layer 230 can be pliable. When the containment member 224 is in an expanded position, e.g., inflated with a fluid in its internal cavity 232 after being introduced into a body, the protective layer 230 can be rigid, thereby helping to prevent a cutting element contained within the containment member 224 from cutting through or otherwise releasing the fluid seal provided by the containment member 224 around the cutting element. The protective layer 230 can be formed from a variety of, preferably biocompatible materials, such as reinforced nylon, Kevlar™, and ultra high molecular weight polyethylene (UHMWPE), e.g., Dyneema™ manufactured by DSM Dyneema of Geleen, The Netherlands.

FIGS. 26-31 show an exemplary embodiment of a morcellator 234 in use. A person skilled in the art will appreciate that the method can have any number of variations and can use any morcellator described herein. The morcellator 234 includes an elongate member or shaft 236 having a handle 238 coupled thereto in the shaft's proximal portion 240 and a containment member 242 coupled thereto in the shaft's distal portion 244. A fluid tube 246, a suction tube 248, and a power cable 250 are also coupled to the morcellator 234 in the shaft's proximal portion 240. The containment member 242 is shown in a collapsed, uninflated, or insertion position where the containment member 242 is rolled around the shaft 236, although in the collapsed position, the containment member 242 can be otherwise positioned such that it can be flush with, e.g., sit or rest upon, or be recessed in the shaft 236.

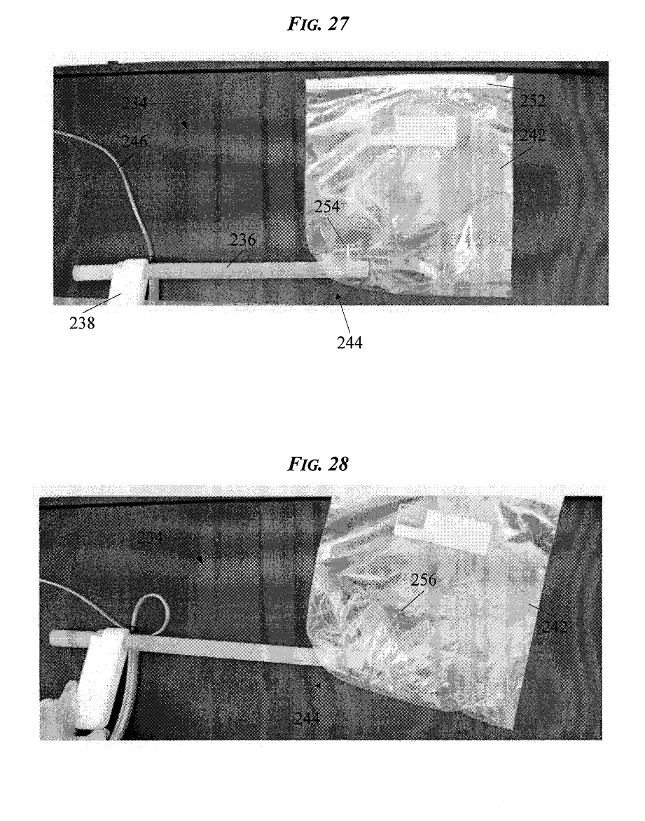

At least the distal portion 244 of the shaft 236 can be introduced into a body through a laparoscopic port (or in any other way) and positioned in a desired location. The containment member 242 can be moved from its insertion position to an expanded or inflated position, shown in FIG. 27, before, or preferably after, the morcellator's shaft 236 has been positioned at or near its desired location. The containment member 242 can be locally expanded or inflated, e.g., by unrolling the containment member 242 using another surgical instrument such as graspers, or the containment member 242 can be remotely expanded or inflated, e.g., by actuating a control at the morcellator's handle 238 to introduce a fluid into the containment member's internal cavity, such as by introducing fluid, preferably a combination of liquid and gas, through at least the fluid tube 246 and out a fluid port in the shaft's distal portion 244. The containment member 242 can be introduced into a body in either an open or closed position, and if in a closed position with a zipper locking seal 252 closed, the containment member 242 can be moved to the open position, such as by locally or remotely opening the zipper locking seal, to prepare the containment member 242 to contain tissue to be macerated by the morcellator's cutting element 254.

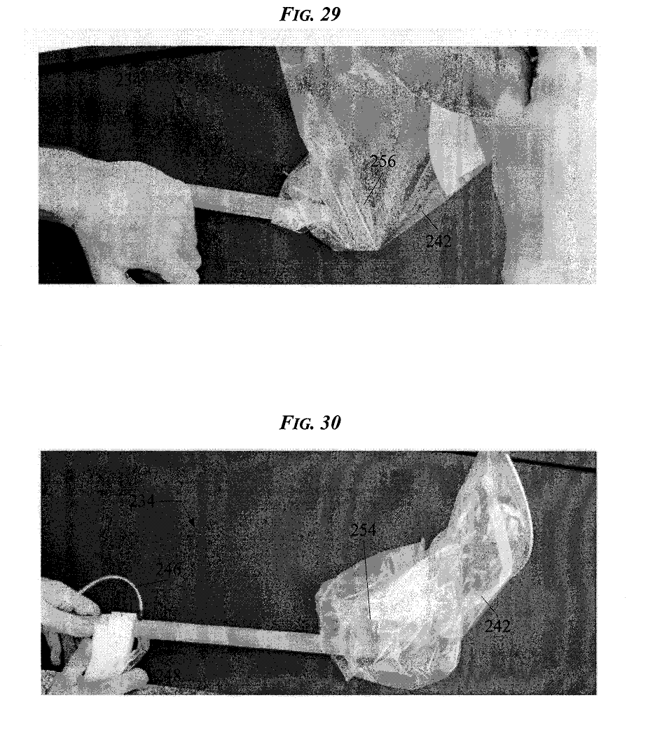

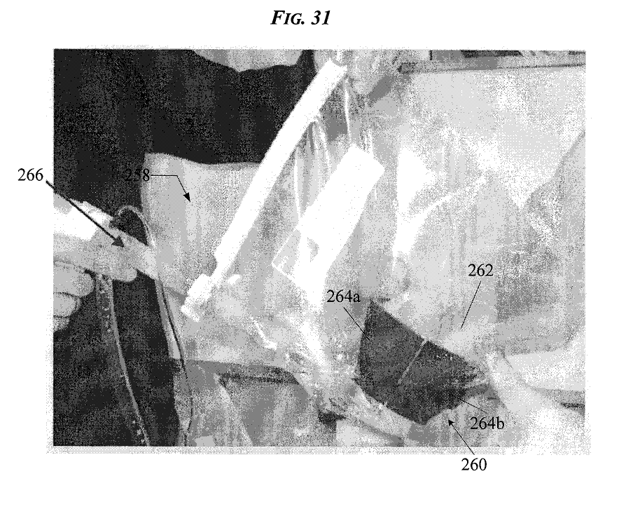

When the containment member 242 is in the open position, as shown in FIG. 28, a tissue 256 can be disposed in the containment member 242 in any way appreciated by a person skilled in the art, such as by maneuvering the tissue using another laparoscopic instrument. Before or after being placed in the containment member 242, the tissue 242 can be separated from other tissue in the body in any laparoscopic way appreciated by a person skilled in the art. The containment member's seal 252 can be moved from the open position to the closed position to provide a substantially fluid tight seal around the tissue 256 and the shaft's distal portion 244. Any amount of fluid can be introduced into the containment member 252 via the fluid tube 246, either continuously or in one or more fluid delivery intervals. The cutting element 254 can be caused to spin and thereby macerate the tissue 256, as shown in FIG. 29, in any way appreciated by a person skilled in the art, such as by actuating a control at the morcellator's handle 238 or by rotating a proximal end (not shown) of the power cable 250. The cutting element 254 can rotate for any amount of time, continuously or in bursts. The tissue 256 can be directed toward the cutting element 254 by gravity, by a guard member (if present), with assistance from one or more other surgical instruments, and/or in any other way appreciated by a person skilled in the art. Suction can be applied to the containment member's internal cavity via the suction tube 248 at any one or more times before, during, or after the cutting element's rotation. When the tissue 256 has been macerated by the cutting element 254 and aspirated through the aspiration tube 248 to an acceptable degree, as shown in FIG. 30 where the containment member's internal cavity is substantially free of tissue and fluid, fluid can cease being supplied through the fluid tube 246 and suction can cease being applied via the suction tube 248. The morcellator 234 can be removed from the body with the containment member 242 coupled thereto, preferably in closed and unexpanded positions.

If, as shown in FIG. 31, a morcellator 258 includes a guard member 260 configured to move between open and closed positions, the guard member 260 is preferably inserted into a body in the closed position and moved to the open position after insertion into a body, either before or after a tissue 262 to be macerated has been positioned proximate to a cutting element (obscured by the tissue 262 and the guard member's arms 264a, 264b) coupled to the morcellator's shaft 266.

One skilled in the art will appreciate further features and advantages of the invention based on the above-described elements. Accordingly, the invention is not to be limited by what has been particularly shown and described, except as indicated by the appended claims. All publications and references cited herein are expressly incorporated herein by reference in their entirety.

What is claimed is:

1. A maceration device, comprising:
an elongate hollow member configured to be at least partially introduced into a body in a minimally invasive surgical procedure; and
a solid cutting element positioned on a side of the elongate hollow member, a longitudinal axis of the cutting element configured to be substantially parallel to an elongate axis of the elongate hollow member when the elongate hollow member and the cutting element are introduced into a body, wherein the cutting element is configured to rotate to macerate tissue.

2. The device of claim 1, wherein a length of the cutting element along the cutting element's longitudinal axis is larger than a largest cross-sectional dimension of a distal end of the elongate hollow member.

3. The device of claim 1, wherein a largest cross-sectional dimension of the distal end of the elongate hollow member is less than about 1 inch.

4. The device of claim 1, wherein a rotational plane of the cutting element and a plane parallel to a cross section of the elongate hollow member are substantially non-parallel.

5. The device of claim 1, wherein the cutting element is substantially flat.

6. The device of claim 1, wherein the cutting element is positioned proximal to a distal end of the elongate hollow member.

7. The device of claim 1, wherein the side of the elongate hollow member includes a recess configured to seat the cutting element therein.

8. The device of claim 1, wherein the cutting element is configured to macerate tissue at a rate greater than about 40 grams per minute.

9. The device of claim 1, further comprising a shaft coupled with the elongate hollow member and configured to deliver power to the cutting element to allow the cutting element to rotate.

10. The device of claim 1, wherein the shaft is rotatably disposed within the elongate hollow member.

11. The device of claim 1, wherein the shaft is detachedly coupled to the elongate hollow member.

12. The device of claim 1, further comprising a tissue containment member configured to enclose the cutting element and at least a distal end of the elongate hollow member when the cutting element and the distal end of the elongate hollow member are disposed in a body, and configured to contain tissue macerated by the cutting element.

13. (canceled)

14. (canceled)

15. (canceled)

16. (canceled)

17. (canceled)

18. (canceled)

19. The device of claim 1, further comprising a rigid guard member configured to at least partially enclose the cutting element when the cutting element rotates.

20. The device of claim 19, wherein the rigid guard member comprises at least two movable arms coupled to the elongate hollow member and configured to be in a closed position substantially flush with the elongate hollow member when the elongate hollow member is introduced into a body and to move to an open position extending out from the elongate hollow body to at least partially enclose the cutting element when the cutting element rotates.

21. The device of claim 19, wherein the rigid guard member comprises a band of synthetic fiber material disposed under the cutting element, wherein a largest diameter of the band of synthetic fiber material is at least as long as a longitudinal length of the cutting element.

22. A maceration device, comprising:
an elongate member having a bore therein, the elongate member configured to be disposed in a body;
a shaft configured to rotate while coupled to the elongate member; and
a substantially flat cutting element coupled to a surface of the elongate member proximal to a distal end of the elongate member, wherein the cutting element is configured to be disposed in a body and to rotate to macerate tissue with power provided by the shaft when the shaft rotates.

23. The device of claim 22, wherein the shaft is removably coupled to the elongate member.

24. The device of claim 22, wherein a longitudinal axis of the cutting element and an elongate axis of the elongate member are configured to be substantially non-parallel during at least a portion of the cutting element's rotation.

25. A maceration device, comprising:
a rigid elongate member configured to be at least partially introduced into a body through an opening having a largest diameter less than about 2 cm; and
a rigid cutting element having a longitudinal length greater than about 2 cm and coupled to the elongate member proximal to a distal end of the elongate member, wherein the cutting element is configured to be introduced into the body through the opening when the elongate member is being at least partially introduced into the body and to rotate to macerate tissue such that a longitudinal axis of the cutting element is not parallel to an elongate axis of the elongate member during at least a portion of the cutting element's rotation.

26. The device of claim 25, further comprising a motor coupled to the elongate member and configured to provide power to the cutting element to allow the cutting element to macerate tissue at a rate of about 50 grams per minute to about 500 grams per minute.
